(19) United States
(12) Patent Application Publication
(10) Pub. No.: US 2008/0226707 A1
(43) Pub. Date: Sep. 18, 2008

(54) WNT compositions and methods of use thereof

(76) Inventors: Jill Helms, Stanford, CA (US); Roeland Nusse, Stanford, CA (US); Jaebeom Kim, Mountain View, CA (US); Philipp Leucht, Woodside, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/074,766

(22) Filed: Mar. 5, 2008

Related U.S. Application Data
(60) Provisional application No. 60/905,282, filed on Mar. 5, 2007.

(51) Int. Cl. A61K 9/127; A61K 38/17; A61P 19/00

(52) U.S. Cl. 424/450; 514/12

(57) ABSTRACT

Methods and compositions are provided for the therapeutic use of Wnt proteins, where the Wnt protein is inserted in the non-aqueous phase of a lipid structure. In some embodiments the Wnt protein is presented in its active conformation on an outer liposome membrane or micelle. Pharmaceutical compositions of the present invention can be administered to an animal for therapeutic purposes. In some embodiments of the invention, the compositions are administered locally, e.g. by injection at the site of an injury. For certain conditions it is desirable to provide Wnt activity for short periods of time, and an effective dose will be administered over a defined, short period of time.

### BACKGROUND OF THE INVENTION

Wnt proteins form a family of highly conserved secreted signaling molecules that regulate cell-to-cell interactions during embryogenesis. Wnt genes and Wnt signaling are also implicated in cancer. Insights into the mechanisms of Wnt action have emerged from several systems: genetics in Drosophila and Caenorhabditis elegans; biochemistry in cell culture and ectopic gene expression in Xenopus embryos. Many Wnt genes in the mouse have been mutated, leading to very specific developmental defects. As currently understood, Wnt proteins bind to receptors of the Frizzled family on the cell surface. Through several cytoplasmic relay components, the signal is transduced to beta-catenin, which then enters the nucleus and forms a complex with TCF to activate transcription of Wnt target genes.

Wnt glycoproteins are thought to function as paracrine or autocrine signals active in several primitive cell types. The Wnt growth factor family includes more than 19 genes identified in the mouse and in humans. The Wnt-1 proto-oncogene (int-1) was originally identified from mammary tumors induced by mouse mammary tumor virus (MMTV) due to an insertion of viral DNA sequence (Nusse and Varmus (1982) Cell 31: 99-109). Expression of Wnt proteins varies, but is often associated with developmental process, for example in embryonic and fetal tissues. Wnts may play a role in local cell signaling. Biochemical studies have shown that much of the secreted Wnt protein can be found associated with the cell surface or extracellular matrix rather than freely diffusible in the medium.

Studies of mutations in Wnt genes have indicated a role for Wnts in growth control and tissue patterning. In Drosophila, wingless (wg) encodes a Wnt gene and wg mutations alter the pattern of embryonic ectoderm, neurogenesis, and imaginal disc outgrowth. In Caenorhabditis elegans, lin-44 encodes a Wnt, which is required for asymmetric cell divisions. Knock-out mutations in mice have shown Wnts to be essential for brain development, and the outgrowth of embryonic primordia for kidney, tail bud, and limb bud. Overexpression of Wnts in the mammary gland can result in mammary hyperplasia, and precocious alveolar development.

Wnt signaling is involved in numerous events in animal development, including the proliferation of stem cells and the specification of the neural crest. Wnt proteins are therefore potentially important reagents in expanding specific cell types, and in treatment of conditions in vivo. The development of pharmaceutically active wnt compositions is therefore of great interest.

### Publications

The biological activity of soluble wingless protein is described in van Leeuwen et al. (1994) Nature 24: 368(6469): 3424. Biochemical characterization of Wnt-frizzled interactions using a soluble, biologically active vertebrate Wnt protein is described by Hsieh et al. (1999) Proc Natl Acad Sci U S A 96(7): 3546-51. Bradley et al. (1995) Mol Cell Biol 15(8): 4616-22 describe a soluble form of wnt protein with mitogenic activity.

### SUMMARY OF THE INVENTION

Methods and compositions are provided for the therapeutic use of Wnt proteins. In some embodiments of the invention, a pharmaceutical composition for in vivo administration is provided, comprising a therapeutically effective dose of a Wnt protein, where the Wnt protein is inserted in the non-aqueous phase of a lipid structure, e.g. in the surface of a liposome, micelle, lipid raft, etc., in an emulsion, and the like. In some embodiments the Wnt protein is presented in its active conformation on an outer liposome membrane or micelle. Where the lipid structure is a liposome it is desirable that the Wnt protein not be encapsulated within the liposome, e.g. in an aqueous phase.

In some embodiments of the invention, the Wnt protein is a mammalian protein, including, without limitation, human Wnt proteins, e.g. Wnt3A. The Wnt compositions find use in a variety of therapeutic methods, including the maintenance and growth of stem cells, tissue regeneration, and the like.

Pharmaceutical compositions of the present invention can be administered to an animal for therapeutic purposes. In some embodiments of the invention, the compositions are administered locally, e.g. by injection at the site of an injury. For certain conditions it is desirable to provide Wnt activity for short periods of time, and an effective dose will be administered over a defined, short period of time.

In some embodiments of the invention, a pharmaceutical composition of the present invention is administered to an animal to accelerate bone repair, e.g. following an injury, in the treatment of bone disease, etc. It is shown herein that a pulse of Wnt activity significantly accelerates bone regeneration by taking advantage of the early Wnt-dependent proliferative effect but avoiding detrimental consequences of persistent Wnt activation. It is also surprisingly found that Wnt in aqueous phase was ineffective in comparison with a formulation where the Wnt protein was inserted in the non-aqueous phase of a lipid structure. In an alternative embodiment for accelerating bone repair, bone marrow cells are contacted with Wnt ex vivo prior to administration of the cells to a patient suffering from a bone injury or disease. This treatment is optionally combined by local administration of a pharmaceutical composition of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Wnt signaling in the intact and injured skeleton. Components of the Wnt pathway, including (a) Wnt3a and (b) Dkk2, are expressed in periosteum (po) and cortical bone (cb) whereas other genes including (c) Wnt2b are expressed solely in periosteum. These expression patterns and others are summarized in Table 1. (d) Pentachrome staining illustrates the organization of the growth plate, where columns of hypertrophic chondrocytes (hc) and trabecular osteoblasts (tb) are interlaced with blood vessels (bv). (e) In the TOPgal growth plate, reporter activity is detectable in chondrocytes and osteoblasts. (f) Reporter activity is also seen in osteocytes of the cortical bone (cb), and (g) at very low levels in the bone marrow. (h) To study the role of Wnt signaling in skeletal repair an injury model is used, which consists of a 1.0 mm hole that penetrates one cortex and enters into the bone marrow (bm) cavity, but leaves the second cortex intact. (i) Newly deposited osteoid matrix, which stains light blue, is readily observable by post-surgical- d6. (j) RTPCR show that in BATgal mice, the reporter beta-galactosidase is expressed within 48 h of injury (red arrow). (k) Immunohistochemical detection of beta-galactosidase by Xgal staining shows that Wnt responsive cells are tightly confined to the injury site at the 72 h post-injury. (I-n) In situ hybridization for components of the Wnt pathway show their widespread expression throughout the injury site. Wnt3a, Dkk2, and Wnt2b showed representative expression patterns; other gene expression patterns are summarized in Table 1. Scale bar: 100 μm.

FIG. 2. Inhibition of Wnt signaling by Dkk1 arrests bone regeneration. TOPgal mice were used for this study, where skeletal injury was generated in the tibia, followed immediately by the systemic administration of a control virus, Ad-Fc and a virus expressing the soluble Wnt inhibitor, Dkk1. (a) Xgal staining illustrates that the pattern of beta-galactosidase reporter activity is unperturbed in the growth plates (gp) of the tibia (ti) and fibula (fi) after systemic delivery of Ad-Fc. (b) Systemic delivery of Ad-Dkk1 nearly abolishes beta-galactosidase activity in the growth plates of TOPgal mice. Insets: Western blots in multiple animals (numbered lanes) confirm that high levels of the Fc fragment and Dkk1 protein are achieved within 48 h of adenoviral delivery. (c) Tissue sections from an Ad-Fc injury site on post-surgical d6 show evidence of new bone matrix, which stains blue (dotted line circumscribes the regenerate). (d) In the Ad-Dkk1 injury site, there is very little new bone (dotted line). The amount of new bone was quantified using histomorphometric measurements (see Methods), which shows that relative to (e) control, Ad-Fc injury sites, (f) Ad-Dkk1 injury sites have 84% drop in bone regeneration. Scale bars: 1 mm (a, b), 100 μm (c, d).

FIG. 3. Bone regeneration is halted because of a Dkk1-mediated arrest in osteoblast differentiation. (a) On post-surgical d4, PECAM immuno-positive endothelial cells fill the Ad-Fc injury site (is), illustrating the typical angiogenic response following skeletal damage. The cut edge of the cortical bone (cb) is circumscribed by a dotted line in all panels. (b) Ad-Dkk1 treatment does not alter PECAM staining to a notable degree. (c) in the Ad-Fc injury site few if any TUNEL-positive cells are detectable; (d) Ad-Dkk1 treatment does not result in a discernable increase in apoptotic cells. (e) Cells in the Ad-Fc injury site begin to differentiate into osteoblasts, as indicated by the widespread expression of runx2. (f) Runx2 expression is undetectable in the Ad-Dkk1 injury site. (g) Ad-Fc injury sites show robust alkaline phosphatase activity, indicating the onset of mineralization of the osteoid matrix. (h) There is no evidence of alkaline phosphatase activity in the injury site of Ad-Dkk1 treated animals; residual alkaline phosphatase is evident on the damaged edges of the cortical bone. Scale bar: 100 μm.

FIG. 4. Constitutive Wnt activation stimulates cell proliferation but delays bone regeneration. (a) Pentachrome staining shows the amount of bone regeneration (dotted line circumscribes new bone) seen in the injury site (is) on post-surgical 6d in wild type animals. (b) Identical injuries in LRP5-G171V mice show no new bone on d6. (c) PCNA immuno-positive cells are present in wild type periosteum (po) adjacent to the injury site; (d) More PCNA-positive cells are present in the injured periosteum of LRP5-G171V mice. (e) PCNA labeled cells are found throughout the wild type bone marrow adjacent to the injury site. (f) LRP5-G171V bone marrow near the injury site shows a dramatic increase in PCNA-positive cells. (g) In situ hybridization for osteochondroprogenitor genes reveals moderate levels of sox9 in the wild type injury site but (h) undetectable expression in the LRP5-G171V injury. (i) Runx2 is also expressed in wild type injury sites but (j) runx2 is expressed at nearly undetectable levels in the LRP5-G171V injury site. (j ) Wild type cells express the osteoblast marker osteocalcin whereas (k) in the LRP5-G171V injury site osteocalcin expression is very low. Scale bar: 100 μm.

FIG. 5. Wnt3a liposomes enhance bone regeneration. (a) SuperTOPflash reporter cells, which express luciferase in response to Wnt activation, were treated with different concentrations of Wnt3a liposomes, PBS liposomes, or Wnt3a protein. After 16 h of incubation, luciferase activity was measured. Wnt3a liposomes activated the reporter in a concentration dependent manner whereas PBS liposomes did not show activity above baseline. (b) Pentachrome staining of a d6 injury site (is) treated with PBS liposomes shows a modest amount of bone regeneration, similar to untreated controls (e.g., FIGS. 1i and 4a). (c) Injury sites that received one injection of Wnt3a liposomes exhibit robust bone regeneration. (d) Bone formation was quantified using Aniline Blue stained tissue sections; injury sites treated with PBS liposome show very little new bone whereas (e) injury sites treated with Wnt3a liposomes show abundant osteoid tissue; histomorphometric measurements revealed a 350% increase in new bone formation at post-surgical d6. (f) Injury sites treated with PBS liposomes show moderate PECAM immunostaining. The cut edge of the cortical bone (cb) is indicated by a dotted line. (g) Injury sites treated with Wnt3a liposomes show an increase in PECAM positive cells (h) Injury sites treated with PBS liposomes show a minimal amount of TRAP activity, due to the small amount of new bone matrix present at this time point; (i) injury sites treated with Wnt3a liposomes show a moderate amount of TRAP activity, indicating that Wnt treatment does not repress osteoclast activity. (j) PBS liposomes elicit only a modest amount of beta-galactosidase activity in the bone marrow near the TOPgal injury site (k) Wnt3a liposomes induce up-regulation of beta-galactosidase activity in the bone marrow near the injury site. (I) Compared to PBS liposomes, (m) Wnt3a liposomes cause an increase in PCNA immunostaining in the injury site. (n) Runx2 is expressed at low levels in the PBS liposome injury sites at d6, whereas (o) strong runx2 expression is seen in Wnt3a-treated injury sites at d6. (p) Sox9 expression was undetectable in PBS-treated injury sites, but (q) sox9 is strongly expressed in the Wnt3a-treated sites. (r) Osteocalcin expression levels are very low in PBS-treated injury sites but (s) osteocalcin is strongly expressed throughout the Wnt3a-treated injury site. Scale bar: 100 μm.

### DESCRIPTION OF THE SPECIFIC EMBODIMENTS

Methods and compositions are provided for the therapeutic use of Wnt proteins. In some embodiments of the invention, a pharmaceutical composition for in vivo administration is provided, comprising a therapeutically effective dose of a Wnt protein, where the Wnt protein is inserted in the non-aqueous phase of a lipid structure, e.g. in the surface of a liposome, micelle, lipid raft, etc., in an emulsion, and the like. In some embodiments the Wnt protein is presented in its active conformation on an outer liposome membrane or micelle. Pharmaceutical compositions of the present invention can be administered to an animal for therapeutic purposes. In some embodiments of the invention, the compositions are administered locally, e.g. by injection at the site of an injury.

In some embodiments of the invention, a pharmaceutical composition of the present invention is administered to an animal to accelerate bone repair, e.g. following an injury, in the treatment of bone disease, etc. In an alternative embodiment for accelerating bone repair, bone marrow cells are contacted with Wnt ex vivo prior to administration of the cells to a patient suffering from a bone injury or disease. This treatment is optionally combined by local administration of a pharmaceutical composition of the invention.

Biologically active Wnt pharmaceutical compositions retain the effector functions that are directly or indirectly caused or performed by native sequence Wnt polypeptides when administered in vivo. Effector functions of native sequence Wnt polypeptides include stabilization of β-catenin, stimulation of stem cell self-renewal, and the like. The Wnt compositions find use in a variety of therapeutic methods, including the maintenance and growth of stem cells, tissue regeneration, and the like.

For use in the above methods, the invention also provides an article of manufacture, comprising: a container, a label on the container, and a composition comprising an active agent within the container, wherein the composition comprises substantially homogeneous biologically active Wnt protein inserted in the non-aqueous phase of a lipid structure, which is effective in vivo, for example in enhancing proliferation and/or maintenance of stem or progenitor cells, and the -label on the container indicates that the composition can be used for enhancing proliferation and/or maintenance of those cells.

### DEFINITIONS

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges encompassed within the invention, subject to any specifically excluded limit in the stated range.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a microsphere” includes a plurality of such microspheres and reference to “the stent” includes reference to one or more stents and equivalents thereof known to those skilled in the art, and so forth.

Wnt protein. Wnt proteins form a family of highly conserved secreted signaling molecules that regulate cell-to-cell interactions during embryogenesis. The terms “Wnts” or “Wnt gene product” or “Wnt polypeptide” when used herein encompass native sequence Wnt polypeptides, Wnt polypeptide variants, Wnt polypeptide fragments and chimeric Wnt polypeptides. In some embodiments of the invention, the Wnt protein comprises palmitate covalently bound to a cysteine residue.

A “native sequence” polypeptide is one that has the same amino acid sequence as a Wnt polypeptide derived from nature. Such native sequence polypeptides can be isolated from cells producing endogenous Wnt protein or can be produced by recombinant or synthetic means. Thus, a native sequence polypeptide can have the amino acid sequence of, e.g. naturally occurring human polypeptide, murine polypeptide, or polypeptide from any other mammalian species, or from non-mammalian species, e.g. Drosophila, C. elegans, and the like.

The term “native sequence Wnt polypeptide” includes human and murine Wnt polypeptides. Human wnt proteins include the following: Wnt 1, Genbank reference NP_{—}005421.1; Wnt 2, Genbank reference NP_{—}003382.1, which is expressed in brain in the thalamus, in fetal and adult lung and in placenta; two isoforms of Wnt 2B, Genbank references NP_{—}004176.2 and NP_{—}078613.1. Isoform 1 is expressed in adult heart, brain, placenta, lung, prostate, testis, ovary, small intestine and colon. In the adult brain, it is mainly found in the caudate nucleus, subthalamic nucleus and thalamus. Also detected in fetal brain, lung and kidney. Isoform 2 is expressed in fetal brain, fetal lung, fetal kidney, caudate nucleus, testis and cancer cell lines. Wnt 3 and Wnt3A play distinct roles in cell-cell signaling during morphogenesis of the developing neural tube, and have the Genbank references NP_{—}110380.1 and X56842. Wnt3A is expressed in bone marrow. Wnt 4 has the Genbank reference NP_{—}110388.2. Wnt 5A and Wnt 5B have the Genbank references NP_{—}003383.1 and AK013218. Wnt 6 has the Genbank reference NP_{—}006513.1; Wnt 7A is expressed in placenta, kidney, testis, uterus, fetal lung, and fetal and adult brain, Genbank reference NP_{—}004616.2. Wnt 7B is moderately expressed in fetal brain, weakly expressed in fetal lung and kidney, and faintly expressed in adult brain, lung and prostate, Genbank reference NP_{—}478679.1. Wnt 8A has two alternative transcripts, Genbank references NP_{—}114139.1 and NP_{—}490645.1. Wnt 8B is expressed in the forebrain, and has the Genbank reference NP_{—}003384.1. Wnt 10A has the Genbank reference NP_{—}079492.2. Wnt 10B is detected in most adult tissues, with highest levels in heart and skeletal muscle. It has the Genbank reference NP_{—}003385.2. Wnt 11 is expressed in fetal lung, kidney, adult heart, liver, skeletal muscle, and pancreas, and has the Genbank reference NP_{—}004617.2. Wnt 14 has the Genbank reference NP_{—}003386.1. Wnt 15 is moderately expressed-in fetal kidney and adult kidney, and is also found in brain. It has the Genbank reference NP_{—}003387.1. Wnt 16 has two isoforms, Wnt-16a and Wnt-16b, produced by alternative splicing. Isoform Wnt-16B is expressed in peripheral lymphoid organs such as spleen, appendix, and lymph nodes, in kidney but not in bone marrow. Isoform Wnt-16a is expressed at significant levels only in the pancreas. The Genbank references are NP_{—}057171.2 and NP_{—}476509.1.

The term “native sequence Wnt protein” includes the native proteins with or without the initiating N-terminal methionine (Met), and with or without the native signal sequence. The native sequence human and murine Wnt polypeptides known in the art are from about 348 to about 389 amino acids long in their unprocessed form reflecting variability (particularly at the poorly conserved amino-terminus and several internal sites), contain 21 conserved cysteines, and have the features of a secreted protein. The molecular weight of a Wnt polypeptide is about 38-42 kD.

A “variant” polypeptide means a biologically active polypeptide as defined below having less than 100% sequence identity with a native sequence polypeptide. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Ordinarily, a biologically active Wnt variant will have an amino acid sequence having at least about 90% amino acid sequence identity with a native sequence Wnt polypeptide, preferably at least about 95%, more preferably at least about 99%.

A “chimeric” Wnt polypeptide is a polypeptide comprising a Wnt polypeptide or portion (e.g., one or more domains) thereof fused or bonded to heterologous polypeptide. The chimeric Wnt polypeptide will generally share at least one biological property in common with a native sequence Wnt polypeptide. Examples of chimeric polypeptides include immunoadhesins, combine a portion of the Wnt polypeptide with an immunoglobulin sequence, and epitope tagged polypeptides, which comprise a Wnt polypeptide or portion thereof fused to a “tag polypeptide”. The tag polypeptide has enough residues to provide an epitope against which an antibody can be made, yet is short enough such that it does not interfere with biological activity of the Wnt polypeptide. Suitable tag polypeptides generally have at least six amino acid residues and usually between about 6-60 amino acid residues.

A “functional derivative” of a native sequence Wnt polypeptide is a compound having a qualitative biological property in common with a native sequence Wnt polypeptide. “Functional derivatives” include, but are not limited to, fragments of a native sequence and derivatives of a native sequence Wnt polypeptide and its fragments, provided that they have a biological activity in common with a corresponding native sequence Wnt polypeptide. The term “derivative” encompasses both amino acid sequence variants of Wnt polypeptide and covalent modifications thereof.

Biologically Active Wnt. The methods of the present invention provide for Wnt compositions that are active when administered to an animal, e.g. a mammal, in vivo. One may determine the specific activity of a Wnt protein in a composition by determining the level of activity in a functional assay after in vivo administration, e.g. accelerating bone regeneration, upregulation of stem cell proliferation, etc., quantitating the amount of Wnt protein present in a non-functional assay, e.g. immunostaining, ELISA, quantitation on coomasie or silver stained gel, etc., and determining the ratio of in vivo biologically active Wnt to total Wnt.

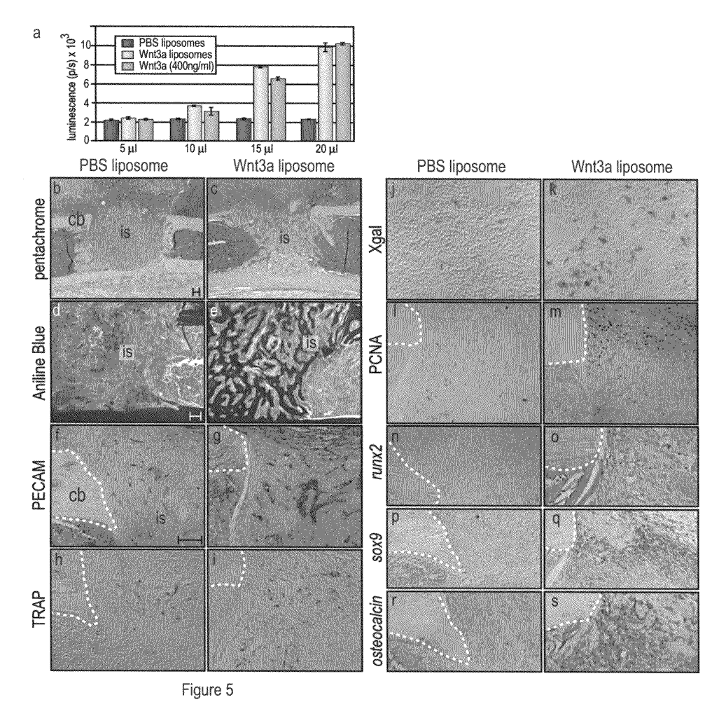

Lipid Structure. As used in the methods of the invention, lipid structures are found to be important in maintaining the activity of wnt proteins following in vivo administration. The wnt proteins are not encapsulated in the aqueous phase of these structures, but are rather integrated into the lipid membrane, and may be inserted in the outer layer of a membrane, as shown in FIG. 5. Such a structure is not predicted from conventional methods of formulating proteins in, for example, liposomes.

Suitable lipids include fatty acids, neutral fats such as triacylglycerols, fatty acid esters and soaps, long chain (fatty) alcohols and waxes, sphingoids and other long chain bases, glycolipids, sphingolipids, carotenes, polyprenols, sterols, and the like, as well as terpenes and isoprenoids. For example, molecules such as diacetylene phospholipids may find use.

Included are cationic molecules, including lipids, synthetic lipids and lipid analogs, having hydrophobic and hydrophilic moieties, a net positive charge, and which by itself can form spontaneously into bilayer vesicles or micelles in water. The term also includes any amphipathic molecules that can be stably incorporated into lipid micelle or bilayers in combination with phospholipids, with its hydrophobic moiety in contact with the interior, hydrophobic region of the micelle or bilayer membrane, and its polar head group moiety oriented toward the exterior, polar surface of the membrane.

The term “cationic amphipathic molecules” is intended to encompass molecules that are positively charged at physiological pH, and more particularly, constitutively positively charged molecules, comprising, for example, a quaternary ammonium salt moiety. Cationic amphipathic molecules typically consist of a hydrophilic polar head group and lipophilic aliphatic chains. Similarly, cholesterol derivatives having a cationic polar head group may also be useful. See, for example, Farhood et al. (1992) Biochim. Biophys. Acta 1111 :239-246; Vigneron et al. (1996) Proc. Natl. Acad. Sci. (USA) 93: 9682-9686.

Cationic amphipathic molecules of interest include, for example, imidazolinium derivatives (WO 95/14380), guanidine derivatives (WO 95/14381), phosphatidyl choline derivatives (WO 95/35301), and piperazine derivatives (WO 95/14651). Examples of cationic lipids that may be used in the present invention include DOTIM (also called BODAI) (Solodin et al., (1995) Biochem. 34: 13537-13544), DDAB (Rose et al., (1991) BioTechniques 10(4): 520-525), DOTMA (U.S. Pat. No. 5,550,289), DOTAP (Eibl and Wooley (1979) Biophys. Chem. 10: 261-271), DMRIE (Felgner et al., (1994) J. Biol. Chem. 269(4): 2550-2561), EDMPC (commercially available from Avanti Polar Lipids, Alabaster, Ala.), DCChol (Gau and Huang (1991) Biochem. Biophys. Res. Comm. 179: 280-285), DOGS (Behr et al., (1989) Proc. Natl. Acad. Sci. USA, 86: 6982-6986), MBOP (also called MeBOP) (WO 95/14651), and those described in WO 97/00241.

While not required for activity, in some embodiments a lipid structure may include a targeting group, e.g. a targeting moiety covalently or non-covalently bound to the hydrophilic head group. Head groups useful to bind to targeting moieties include, for example, biotin, amines, cyano, carboxylic acids, isothiocyanates, thiols, disulfides, α-halocarbonyl compounds, α,β-unsaturated carbonyl compounds, alkyl hydrazines, etc.

Chemical groups that find use in linking a targeting moiety to an amphipathic molecule also include carbamate; amide (amine plus carboxylic acid); ester (alcohol plus carboxylic acid), thioether (haloalkane plus sulfhydryl; maleimide plus sulfhydryl), Schiffs base (amine plus aldehyde), urea (amine plus isocyanate), thiourea (amine plus isothiocyanate), sulfonamide (amine plus sulfonyl chloride), disulfide; hyrodrazone, lipids, and the like, as known in the art.

For example, targeting molecules may be formed by converting a commercially available lipid, such as DAGPE, a PEG-PDA amine, DOTAP, etc. into an isocyanate, followed by treatment with triethylene glycol diamine spacer to produce the amine terminated thiocarbamate lipid which by treatment with the para-isothiocyanophenyl glycoside of the targeting moiety produces the desired targeting glycolipids. This synthesis provides a water soluble flexible linker molecule spaced between the amphipathic molecule that is integrated into the nanoparticle, and the ligand that binds to cell surface receptors, allowing the ligand to be readily accessible to the protein receptors on the cell surfaces.

A targeting moiety, as used herein, refers to all molecules capable of specifically binding to a particular target molecule and forming a bound complex as described above. Thus the ligand and its corresponding target molecule form a specific binding pair.

The term “specific binding” refers to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

Examples of targeting moieties include, but are not limited to antibodies, lymphokines, cytokines, receptor proteins such as CD4 and CD8, solubilized receptor proteins such as soluble CD4, hormones, growth factors, peptidomimetics, synthetic ligands, and the like which specifically bind desired target cells, and nucleic acids which bind corresponding nucleic acids through base pair complementarity. Targeting moieties of particular interest include peptidomimetics, peptides, antibodies and antibody fragments (e.g. the Fab′ fragment). For example, β-D-lactose has been attached on the surface to target the aloglysoprotein (ASG) found in liver cells which are in contact with the circulating blood pool.

Cellular targets include tissue specific cell surface molecules, for targeting to specific sites of interest, e.g. neural cells, liver cells, bone marrow cells, kidney cells, pancreatic cells, muscle cells, and the like. For example, nanoparticles targeted to hematopoietic stem cells may comprise targeting moieties specific for CD34, ligands for c-kit, etc. Nanoparticles targeted to lymphocytic cells may comprise targeting moieties specific for a variety of well known and characterized markers, e.g. B220, Thy-1, and the like.

The use of liposomes or micelles as a delivery vehicle is one method of interest. A liposome is a spherical vesicle with a membrane composed of a phospholipid bilayer. Liposomes can be composed of naturally-derived phospholipids with mixed lipid chains (like egg phosphatidylethanolamine), or of pure surfactant components like DOPE (dioleolylphosphatidylethanolamine). Liposomes often contain a core of encapsulated aqueous solution; while lipid spheres that contain no aqueous material are referred to as micelles. As the wnt proteins are present in the lipid phase and not the encapsulated aqueous phase, micelles may be used interchangeably with liposome for the compositions of the present invention. The lipids may be any useful combination of known liposome or micelle forming lipids, including cationic lipids, such as phosphatidylcholine, or neutral lipids, such as cholesterol, phosphatidyl serine, phosphatidyl glycerol, and the like.

In another embodiment, the vesicle-forming lipid is selected to achieve a specified degree of fluidity or rigidity, to control the stability of the structure in serum, etc. Liposomes having a more rigid lipid bilayer, or a liquid crystalline bilayer, are achieved by incorporation of a relatively rigid lipid, e.g., a lipid having a relatively high phase transition temperature, e.g., up to 60° C. Rigid, i.e., saturated, lipids contribute to greater membrane rigidity in the lipid bilayer. Other lipid components, such as cholesterol, are also known to contribute to membrane rigidity in lipid bilayer structures. Lipid fluidity is achieved by incorporation of a relatively fluid lipid, typically one having a lipid phase with a relatively low liquid to liquid-crystalline phase transition temperature, e.g., at or below room temperature.

The liposomes may be prepared by a variety of techniques, such as those detailed in Szoka, F., Jr., et al., Ann. Rev. Biophys. Bioeng. 9: 467 (1980). Typically, the liposomes are multilamellar vesicles (MLVs), which can be formed by simple lipid-film hydration techniques. In this procedure, a mixture of liposome-forming lipids of the type detailed above dissolved in a suitable organic solvent is evaporated in a vessel to form a thin film, which is then covered by an aqueous medium. The lipid film hydrates to form MLVs, typically with sizes between about 0.1 to 10 microns.

The liposomes micelles, etc. of the invention may have substantially homogeneous sizes in a selected size range, typically between about 0.01 to 0.5 microns, more preferably between 0.03-0.40 microns. One effective sizing method for REVs and MLVs involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less.

A variety of thermosensitive liposomes are also known in the art. For example, a liposome consisting of anionic detergent and phospholipid which releases drugs rapidly at a temperature range of 40° to 45° C., and a liposome consisting of dipalmitoylphosphatidylcholine (DPPC) and 1,2-diacylglycerophospholipid which releases drugs effectively at a temperature range of 40° to 44° C., have been reported (see: Japanese unexamined patent publication (Hei) 06-227966). Kono et al. teaches a thermosensitive liposome which starts to release drugs at a temperature range of 25° to 30° C., i.e., lecithin or DPPC-containing liposome coated with copolymer of N-isopropylacrylamide/octadecylacrylate (see: K. Kono et al., J. Controlled Release, 30: 69-75 (1994)).

The pharmaceutical compositions of the present invention also comprise a pharmaceutically acceptable carrier. Many pharmaceutically acceptable carriers may be employed in the compositions of the present invention. Generally, normal saline will be employed as the pharmaceutically acceptable carrier. Other suitable carriers include, e.g., water, buffered water, 0.4% saline, 0.3% glycine, and the like, including glycoproteins for enhanced stability, such as albumin, lipoprotein, globulin, etc. These compositions may be sterilized by conventional, well known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc.

The concentration of lipid structures in the carrier may vary. Generally, the concentration will be about 0.1 to 1000 mg/ml, usually about 1-500 mg/ml, about 5 to 100 mg/ml, etc. Persons of skill may vary these concentrations to optimize treatment with different lipid components or of particular patients.

Compositions will comprise a therapeutically effective in vivo dose of a wnt protein, and may comprise a cocktail of one or more wnt proteins.

### Therapeutic Methods

The subject methods are useful for both prophylactic and therapeutic purposes. Thus, as used herein, the term “treating” is used to refer to both prevention of disease, and treatment of a pre-existing condition. The treatment of ongoing disease, to stabilize or improve the clinical symptoms of the patient, is a particularly important benefit provided by the present invention. Such treatment is desirably performed prior to loss of function in the affected tissues; consequently, the prophylactic therapeutic benefits provided by the invention are also important. Evidence of therapeutic effect may be any diminution in the severity of disease. The therapeutic effect can be measured in terms of clinical outcome or can be determined by immunological or biochemical tests. Patents for treatment may be mammals, e.g. primates, including humans, may be laboratory animals, e.g. rabbits, rats, mice, etc., particularly for evaluation of therapies, horses, dogs, cats, farm animals, etc.

The dosage of the therapeutic formulation will vary widely, depending upon the nature of the condition, the frequency of administration, the manner of administration, the clearance of the agent from the host, and the like. The initial dose can be larger, followed by smaller maintenance doses. The dose can be administered as infrequently as weekly or biweekly, or more often fractionated into smaller doses and administered daily, semi-weekly, or otherwise as needed to maintain an effective dosage level.

In some embodiments of the invention, administration of the wnt pharmaceutical formulation is performed by local administration. Local administration, as used herein, may refer to topical administration, but more often refers to injection or other introduction into the body at a site of treatment. Examples of such administration include intramuscular injection, subcutaneous injection, intraperitoneal injection, and the like. It is found that the lipid structures of the present invention generally are less effective when systemically administered, and the highest activity may be found at or around the site where it is initially introduced.

In some embodiments of the invention, the formulations are administered on a short term basis, for example a single administration, or a series of administration performed over, e.g. 1, 2, 3 or more days, up to 1 or 2 weeks, in order to obtain a rapid, significant increase in activity. The size of the dose administered must be determined by a physician and will depend on a number of factors, such as the nature and gravity of the disease, the age and state of health of the patient and the patient's tolerance to the drug itself.

For example, a number of conditions are characterized by an inability to regenerate tissues, where upregulation of stem cell activity is desirable.

The term stem cell is used herein to refer to a mammalian cell that has the ability both to self-renew, and to generate differentiated progeny (see Morrison et al. (1997) Cell 88: 287-298). Generally, stem cells also have one or more of the following properties: an ability to undergo asynchronous, or symmetric replication, that is where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages. “Progenitor cells” differ from stem cells in that they typically do not have the extensive self-renewal capacity, and often can only regenerate a subset of the lineages in the tissue from which they derive, for example only lymphoid, or erythroid lineages in a hematopoietic setting.

Stem cells may be characterized by both the presence of markers associated with specific epitopes identified by antibodies and the absence of certain markers as identified by the lack of binding of specific antibodies. Stem cells may also be identified by functional assays both in vitro and in vivo, particularly assays relating to the ability of stem cells to give rise to multiple differentiated progeny.

Stem cells of interest include muscle satellite cells; hematopoietic stem cells and progenitor cells derived therefrom (U.S. Pat. No. 5,061,620); neural stem cells (see Morrison et al. (1999) Cell 96: 737-749); embryonic stem cells; mesenchymal stem cells; mesodermal stem cells; liver stem cells, etc.

The cells of interest are typically mammalian, where the term refers to any animal classified as a mammal, including humans, domestic and farm animals, and zoo, laboratory, sports, or pet animals, such as dogs, horses, cats, cows, mice, rats, rabbits, etc. Preferably, the mammal is human.

In many clinical situations, the bone healing condition are less ideal due to decreased activity of bone forming cells, e.g. within aged people, following injury, in osteogenesis imperfecta, etc. Within bone marrow stroma there exists a subset of non-hematopoietic cells capable of giving rise to multiple cell lineages. These cells termed as mesenchymal stem cells (MSC) have potential to differentiate to lineages of mesenchymal tissues including bone, cartilage, fat, tendon, muscle, and marrow stroma.

A variety of bone and cartilage disorders affect aged individuals. Such tissues are normally regenerated by mesenchymal stem cells. Included in such conditions is osteoarthritis. Osteoarthritis occurs in the joints of the body as an expression of “wear-and-tear”. Thus athletes or overweight individuals develop osteoarthritis in large joints (knees, shoulders, hips) due to loss or damage of cartilage. This hard, smooth cushion that covers the bony joint surfaces is composed primarily of collagen, the structural protein in the body, which forms a mesh to give support and flexibility to the joint. When cartilage is damaged and lost, the bone surfaces undergo abnormal changes. There is some inflammation, but not as much as is seen with other types of arthritis. Nevertheless, osteoarthritis is responsible for considerable pain and disability in older persons.

In conditions of the aged where repair of mesenchymal tissues is decreased, or there is a large injury to mesenchymal tissues, the stem cell activity may be enhanced by administration of tissue regenerating agent(s).

In methods of accelerating bone repair, a pharmaceutical wnt composition of the present invention is administered to a patient suffering from damage to a bone, e.g. following an injury. The formulation is preferably administered at or near the site of injury, following damage requiring bone regeneration. The wnt formulation is preferably administered for a short period of time, and in a dose that is effective to increase the number of bone progenitor cells present at the site of injury. In some embodiments the wnt is administered within about two days, usually within about 1 day of injury, and is provided for not more than about two weeks, not more than about one week, not more than about 5 days, not more than about 3 days, etc.

In an alternative method, patient suffering from damage to a bone is provided with a composition comprising bone marrow cells, e.g. a composition including mesenchymal stem cells, bone marrow cells capable of differentiating into osteoblasts; etc. The bone marrow cells may be treated ex vivo with a pharmaceutical composition comprising a wnt protein or proteins in a dose sufficient to enhance regeneration; or the cell composition may be administered to a patient in conjunction with a wnt formulation of the invention.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Due to biological functional equivalency considerations, changes can be made in protein structure without affecting the biological action in kind or amount. All such modifications are intended to be included within the scope of the appended claims.

### Experiental

### EXAMPLE 1

### Enhanced Bone Regeneration via Wnt3a Liposomes

Some adult tissues have the ability to regenerate and among these, bone is one of the most remarkable. Bone exhibits a persistent, lifelong capacity to re-form following injury, and continual bone regeneration is a prerequisite to maintaining bone mass and density. Even slight perturbations in bone regeneration can have profound consequences, as exemplified by conditions such as osteoporosis and delayed skeletal repair. Here, our goal was to determine the role of Wnts in adult bone formation and then use this information, plus novel reagents, in a therapeutic strategy to stimulate bone regeneration. Using TOPgal reporter mice we found that damage to the skeleton instigated Wnt signaling, specifically at the site of injury. We used a skeletal injury model to determine that Wnt inhibition prevented the differentiation of osteoprogenitor cells and as a result, injury-induced bone regeneration was reduced by 84%. Constitutive activation of the Wnt pathway results in high bone mass, but this same point mutation caused a delay in bone regeneration because osteoprogenitor cells were maintained in a proliferative state. In a therapeutic strategy to enhance bone regeneration we transiently activated Wnt signaling at the injury site. This was accomplished by packaging Wnt3a protein into liposomal vesicles, which preserved the biological activity of the protein in the wound milieu. We found that a single injection of Wnt3a liposomes resulted in 350% increase in bone regeneration, compared to controls. These data demonstrate a useful approach to treating clinical conditions where bone regeneration is desired.

Here, we investigate a regenerative pathway regulated by Wnt proteins. Wnt ligands are secreted molecules that bind to cell surface receptors encoded by the Frizzled and low-density lipoprotein receptor (LRP)-related proteins. Once bound, the ligands initiate a cascade of intracellular events that eventually lead to the transcription of target genes through the nuclear activity of beta-catenin and the DNA binding protein TCF.

Wnts are involved in a wide variety of cellular decisions associated with the program of osteogenesis. For example, Wnts regulate the expression level of Sox9, which influences the commitment of mesenchymal progenitor cells to a skeletogenic fate. Wnts influence the differentiation of cells, into either osteoblasts or chondrocytes. There is also evidence that Wnt signaling regulates bone mass. For example, mutations in the human Wnt coreceptor LRP5 are associated with several high bone mass syndromes including van Buchem disease, osteopetrosis type I, and endosteal hyperostosis or autosomal dominant osteosclerosis, as well as a low bone mass disease, osteoporosis-pseudoglioma (Cheung et al. (2006) Bone 39, 470-6; Kwee et al. (2005) J Bone Miner Res 20, 1254-60; Henriksen et al. (2005) Am J Pathol 167, 1341-8; Van Wesenbeeck et al. (2003) Am J Hum Genet 72, 763-71). Increased production of the Wnt inhibitor DKK1 is associated with multiple myeloma, a disease which has as one of its distinguishing features increased bone resorption. Despite this association between. Wnt signaling and bone disease, it has been difficult to identify how perturbations in this pathway culminate in aberrant bone remodeling.

Unlike the life-long process of bone remodeling, injury-induced bone regeneration occurs within a compressed time frame and in a precise location. The cellular and molecular machinery, however, is identical between bone remodeling and bone regeneration. When an animal sustains skeletal damage, the basic challenge it faces is to detect the injury and then heal the defect as rapidly as possible. This is achieved by the recruitment of skeletal progenitor cells to the injury site; once a sufficient population of progenitor cells has been generated, cells decelerate their proliferation and differentiate into osteoblasts, which secrete a matrix which becomes mineralized. Osteoclasts then remodel the matrix and thus restore the original shape of the bone. In a murine model this entire process, from initial injury to new bone formation, takes less than a week (Colnot et al. (2005) Clin Orthop Relat Res, 69-78).

We exploited the spatiotemporal parameters .of a skeletal injury model to gain an understanding of the function of Wnt signaling in bone regeneration. Here, we report that damage to the skeleton up-regulates Wnt signaling specifically at the injury site, and prompts bone marrow-derived cells to respond to this endogenous Wnt signal. The data show that when Wnt signaling is inhibited then bone marrow-derived cells in the injury site do not differentiate into osteoblasts and bone regeneration is terminated. When Wnt signaling is constitutively activated then cells in the injury site show heightened proliferation but once again do not differentiate into osteoblasts and bone regeneration is temporarily delayed. Based on these observations we formulated a therapeutic strategy to accelerate bone regeneration, which took advantage of the early Wnt-dependent proliferative effect but avoided the detrimental consequences of persistent Wnt activation. Our strategy involved the insertion of Wnt3a protein into the lipid structure of liposomes, spherical nano-vesicles consisting of an aqueous core enclosed in one or more phospholipid layers (reviewed by Banerjee (2001) J Biomater Appl 16, 3-21). We found that Wnt3a liposomes provoked the rapid differentiation of bone marrow-derived cells into osteoblasts, with the result being a dramatic enhancement in bone regeneration.

In sum, these data extend our understanding of Wnt functions in the process of bone regeneration. They also show that the Wnt pathway is a compelling target for therapeutic interventions that seek to stimulate bone formation following disease or injury. In addition, our data show that liposomal packaging is a viable delivery vehicle for Wnts, which advocates their use in a wide variety of clinical applications.

### Results

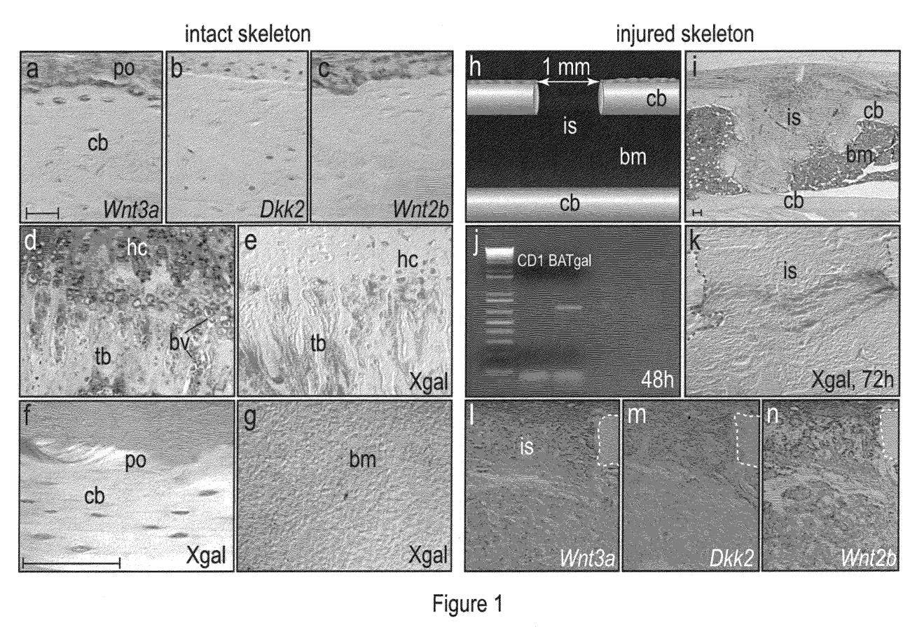

Damage to the skeleton up-regulates Wnt signaling at the site of injury. To gain a better appreciation for which Wnts are involved in the process of bone regeneration we initiated an in situ hybridization-based screening for components of the Wnt pathway. We first examined gene expression in the intact, adult skeleton and found that most components of the pathway were detected at low levels in the growth plate and the periosteum (e.g., FIG. la-c). For example, osteocytes expressed Wnts 3a, 5a, 5b, 11, Frizzled 4 (Fzd4), and Dickkopf2 (Dkk2) whereas cells in the endosteum and bone marrow expressed Wnts 2b, 3a, 5a, 11, Dkk2, and Wnt Inhibitory Factor (FIG. 1a-c and summarized in Table 1). These data were consistent with a role for Wnt signaling in the maintenance of adult bone mass and in a variety of bone diseases. We also examined the intact skeleton of TOPgal and BATgal transgenic mice. These mice carry a beta-galactosidase reporter under the control of multiple copies of the Tcf/Lef binding site; thus, the spatiotemporal pattern of Xgal staining is a reflection of Wnt responsiveness. In adult growth plate, hypertrophic chondrocytes are organized in rows that adjoin trabecular osteoblasts, which in turn are interlaced with blood vessels (FIG. 1d). Both hypertrophic chondrocytes and trabecular osteoblasts in the TOPgal growth plate were Xgal positive (FIG. 1e). Osteocytes embedded in the bone cortex also stained for Xgal (FIG. 1f), as did a small percentage of cells in the bone marrow (FIG. 1g). These data indicated that Wnt signaling was functional in the adult skeleton.

TABLE 1
summary of Wnt signalling in the intact & injured skeleton

| | EXPRESSION DOMAIN | | | | | |
|---|---|---|---|---|---|---|
| | | | | BONE MARROW | | |
| | | | | injury | adjacent | GROWTH PLATE |
| GENE | PERIOSTEUM | OSTEOCYTES | ENDOSTEUM | site | to injury | CHONDROCYTES |
| Wnt 2b | + | − | + | + | + | + |
| Wnt 3a | + | + | + | + | + | + |
| Wnt 7a | + | − | − | + | − | − |
| Wnt 5a | + | + | + | + | + | + |
| Wnt 5b | + | + | − | + | − | + |
| Wnt 11 | + | + | + | + | + | + |
| Frizzled 4 | + | + | − | + | − | + |
| FrzB | + | − | − | − | − | + |
| Dickkopf 2 | + | + | + | + | + | + |
| Wnt inhibitory | + | − | + | + | + | + |
| factor | | | | | | |

Our next objective was to determine the relationship between Wnt signaling and bone remodeling. We found that all of the cellular events that comprise bone remodeling are elaborated when we generate a simple skeletal defect consisting of a 1.0 mm hole drilled through a single cortex of the tibia (FIG. 1h). This type of skeletal defect heals by the recruitment of skeletal progenitor cells to the injury site, followed by their direct differentiation into osteoblasts and the deposition of a mineralized matrix. Osteoclasts then remodel the bony matrix until the shape of the tibia is restored. Bone regeneration occurs exclusively through intramembranous ossification, and new bone is readily detectable in the injury site by post-surgical d6 (FIG. 1i). We generated these tibial defects in TOPgal and BATgal mice and then examined the wound site for evidence of Wnt reporter activity:

The early injury callus is primarily composed of a fibrin clot, whose amorphous material properties complicate tissue sectioning and histological assessment. Therefore, we isolated RNA 24 h and 48 h injury calluses from BATgal mice and performed RT-PCR for expression of the β-galactosidase reporter. Within the first 24 h of skeletal injury, β-galactosidase expression was dramatically increased over baseline, and it remained elevated in the 48 h injury callus (FIG. 1j). By 72 h post-injury, the callus becomes more organized and can be analyzed in tissue sections. We found that reporter activity was restricted to bone marrow-derived cells in the injury site (FIG. 1k). We used in situ hybridization to determine which Wnts were responsible for triggering reporter activity in the BATgal and TOPgal mice, and found that multiple Wnt ligands, co-factors, antagonists, and receptors were expressed throughout the injury site, in the adjacent periosteum, and along the endosteal surface (FIG. 1i-n and Table 1).

Inhibiting Wnt signaling in vivo. Our data demonstrated that components of the Wnt pathway are expressed in the right time and place to play a role in bone regeneration, since Wnt signaling is up-regulated within 24 h of damage to the skeleton; and Wnt responsive cells are localized to the injury site (FIG. 1). Given these data, we tested whether perturbations in Wnt signaling affected the program of skeletal repair.

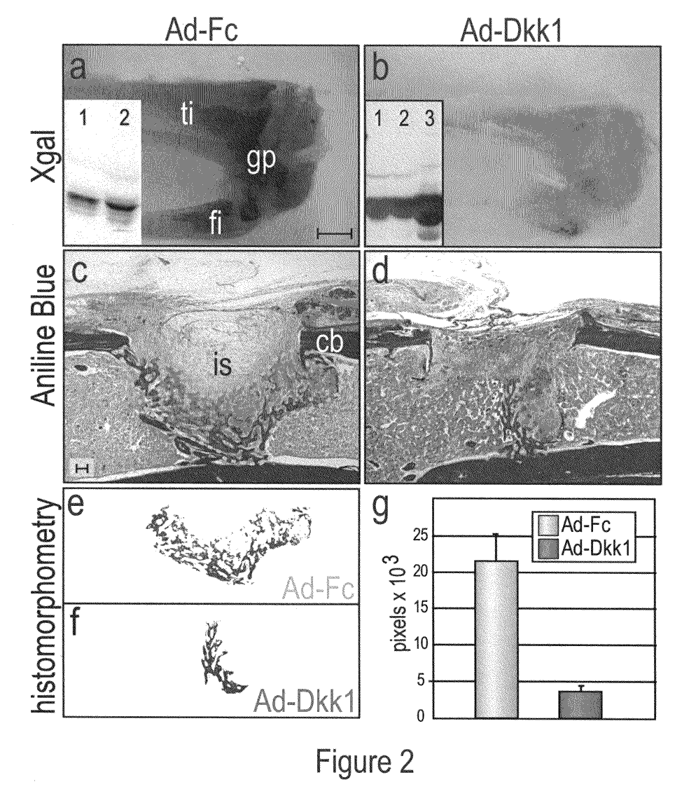

To inhibit Wnt signaling in vivo we injected adenovirus expressing the soluble Wnt antagonist Dkk1 (Ad-Dkk1), into TOPgal mice. This technique produces a conditional, reversible inhibition in Wnt signaling in the adult animal. Adenovirus expressing the Fc portion of mouse immunoglobulin gene (Ad-Fc) served as a control. Immediately after infection, skeletal defects were generated and two days later, we confirmed that high levels of Dkk1 and Fc fragment were being produced (n=6 for each condition; FIG. 2a, b insets).

On post-surgical d6, the injured tibiae were collected from Ad-Fc and Ad-Dkk1 treated TOPgal mice. Whole mount Xgal staining showed that Ad-Dkk1 treatment effectively reduced Wnt signaling throughout the skeleton and in the injury site (FIG. 2a, b). We used histology (FIG. 2c, d) and histomorphometric measurements (FIG. 2e-g) to assess the amount of bone regeneration, and found that Ad-Dkk1 treatment reduced bone regeneration by 84% (control Ad-Fc, n=7; Ad-Dkk1, n=10).

Because Ad-Dkk1 has deleterious systemic effects that may indirectly affect the reparative process, we also tested whether local delivery of Ad-Dkk1 blocked bone regeneration. In these cases Ad-Dkk1 or Ad-Fc was injected into the musculature surrounding the tibia and 48 h, a skeletal defect was generated in this location. Histomorphometric measurements confirmed that local Ad-Dkk1 blocked bone regeneration as effectively as systemic Ad-Dkk1 injection (FIG. 6).

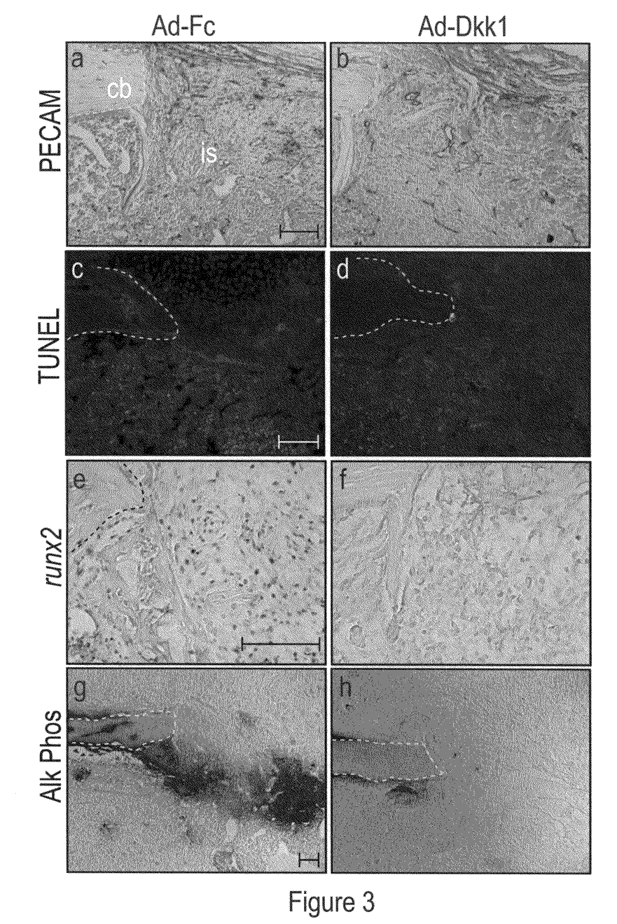

Our next series of experiments were aimed at determining the mechanisms underlying the Dkk-1 mediated arrest in bone regeneration. In previous studies we have shown that a disruption in angiogenesis can delay bone regeneration. When we evaluated endothelial cell invasion into the injury site, however, we did not detect any differences in the extent or distribution of platelet endothelial cell adhesion molecule (PECAM) immunostaining (Ad-Fc, n=4; Ad-Dkk1, n=4; FIG. 3a, b). Therefore, Ad-Dkk1 treatment did not appear to curtail injury-induced angiogenesis. We also used TUNEL staining to evaluate Ad-Dkk1 injury sites, reasoning that Wnt inhibition might lead to an increase in programmed cell death. Once again, Ad-Dkk1 and Ad-Fc tibiae showed equivalent TUNEL staining (Ad-Fc, n=4; Ad-Dkk1, n=4; FIG. 3c, d). Therefore it was unlikely that Ad-Dkk1 hampered bone regeneration by increasing cell death in the injury site.

We examined cells in the Ad-Dkk1 and Ad-Fc injury sites for evidence of osteoblast differentiation. On post-surgical d4, cells in the Ad-Fc injury site strongly expressed the osteogenic genes runx2 and collagen type I whereas in Ad-Dkk1 injury sites, runx2 expression was essentially undetectable (FIG. 3e, f. We also found Ad-Fc injury sites showed robust alkaline phosphatase activity, whereas this same activity that characterizes osteoblast differentiation was all but nonexistent in Ad-Dkk1 injury sites (FIG. 3g, h). Together, these data demonstrated that Dkk1-mediated Wnt inhibition blocked osteoblast differentiation and as a consequence, arrested the program of adult bone regeneration.

Constitutive Wnt activation increases skeletal progenitor cell proliferation but delays bone regeneration. Having demonstrated that inhibition of Wnt signaling was detrimental to bone regeneration we next explored whether activation of Wnt signaling would be advantageous to bone healing. A gain-of function mutation in the Wnt co-receptor Lrp5 is associated with a high bone mass phenotype therefore we evaluated bone regeneration in transgenic mice harboring the same mutation (i.e., Lrp5-G171V mice).

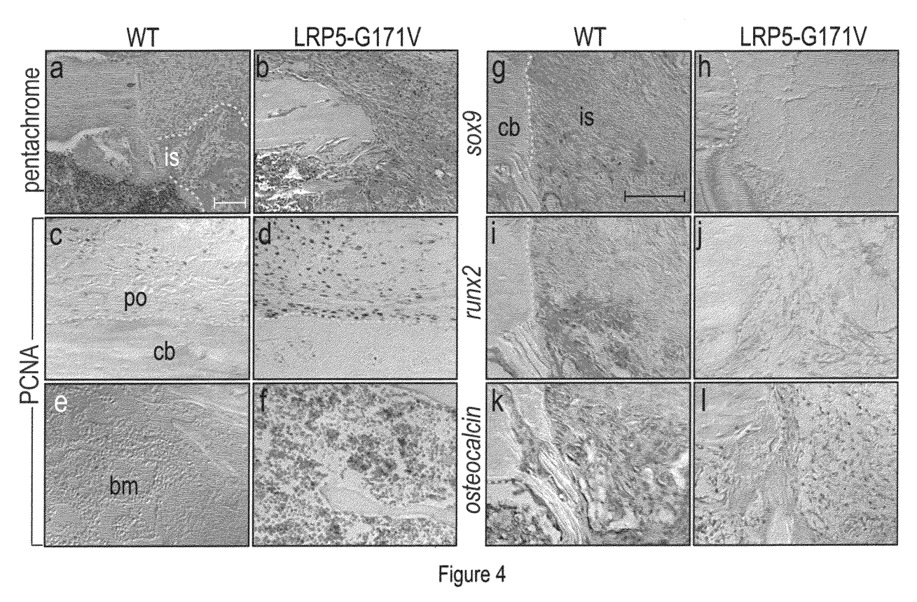

Skeletal defects were generated and samples were analyzed on post-surgical d6. The first and most obvious phenotype we found was the absence of bone regeneration in the Lrp5 injury sites (wild type, n=6 Lrp5-G171V, n=6; FIG. 4a, b). We evaluated cell proliferation using immunohistochemical detection of proliferating cell nuclear antigen (PCNA), and found that in Lrp5-G171V mice, immunostaining was consistently elevated in the periosteum, the injury site, and throughout the bone marrow (FIG. 4c-f. In addition, the expression levels of the pro-osteogenic genes sox9, runx2 and osteocalcin were all reduced in Lrp5 injury sites, relative to wild type littermates (FIG. 4g-l). These data indicated that constitutively active Wnt signaling promoted cell proliferation and prevented cell differentiation in the skeletal injury site. When we examined Lrp5-G171V mice on post-surgical d14, the amount of bone regeneration was equivalent between Lrp5-G171V and wild types. Thus, constitutive activation of the Wnt pathway caused a delay in bone regeneration, by temporarily inhibiting the differentiation of cells into osteoblasts.

In vivo delivery of Wnt3a enhances bone regeneration. Our objective at the outset of these experiments was to gain insights into the role(s) of Wnt signaling in adult bone formation and remodeling, and then use this information to develop therapeutic strategies to enhance bone regeneration. We recognized that the Wnt-dependent proliferative effect in Lrp5-G171V mice can be used to our advantage, and therefore embarked on a series of experiments where we examined the effects of exogenous Wnt protein on bone regeneration. The most challenging technical hurdle we faced was devising an appropriate delivery vehicle for Wnt proteins. In our initial attempts we found that Wnt3a protein directly injected into the injury site had no effect on bone regeneration compared to PBS injection. We also incorporated Wnt3a protein into fibrin glue that was implanted into the injury site. As with direct injection of the protein, we did not detect any differences in onset, rate, or extent of bone regeneration.

We knew that Wnts contain a palmitate residue that is required for in vivo activity, and that Wnts and other lipid modified proteins may remain associated with a membrane even when they are being shuttled between cells. We reasoned that if the endogenous mechanism for transporting lipidated Wnts to their target cells involved an association with lipid vesicle or raft then a technique that commandeered this lipid encapsulation might be especially useful in vivo.

Liposomes are spherical nano-vesicles consisting of an aqueous core enclosed in one or more phospholipid layers. Liposomes were developed in an attempt to improve the pharmocokinetics and tissue distribution of chemotherapeutic agents. We decided to exploit these properties for the in vivo delivery of Wnt3a and therefore tested whether Wnt3a retained its biological activity when packaged into liposomes.

We prepared liposomal vesicles containing Wnt3a and tested them in an in vitro assay. SuperTOPflash cells express luciferase under the control of a promoter containing Tcf/Lef binding sites. We exposed SuperTOPflash cells to Wnt3a protein (positive control), Wnt3a liposomes, or PBS liposomes (negative control). After 16-18 h of incubation luciferase reporter activity was assessed. We found that Wnt3a protein and Wnt3a liposomes both stimulated reporter activity in a concentration dependent manner (n=7 for each condition; FIG. 5a). PBS liposomes elicited only baseline activity, equivalent to PBS alone (FIG. 5a). These results indicated that Wnt3a retained biological activity after liposomal preparation and packaging.

Our next objective was to test the in vivo activity of Wnt3a liposomes. We generated skeletal injuries in TOPgal mice and on post-surgical d3, delivered Wnt3a liposomes or control (PBS) liposomes to the injury site. After 72 h we examined the tissues. Injuries treated with control liposomes exhibited a modest amount of bone regeneration (n=6; FIG. 5b), similar to untreated (normal) skeletal injuries (e.g., FIG. 1i). By contrast, injury sites that received a single injection of Wnt3a liposomes were completely filled with mature bony trabeculae (n=7; FIG. 5c). This dramatic effect was achieved in 72 h, since liposomes were injected on post-surgical d3 and histological evaluations were carried out on post-surgical d6. Histomorphometric analyses showed that injury sites treated with Wnt3a liposomes had 350% more bone than control injury sites (average number of pixels representing bone in Wnt3a treated samples=78146; PBS samples=20615; FIG. 5d, e).

We investigated the basis for this pro-osteogenic effect. Because Wnt3a can induce endothelial cell proliferation and angiogenesis we examined injury sites for evidence of increased PECAM immunostaining. We found a qualitative increase in the number of PECAM positive cells in Wnt3a treated sites (compare control FIG. 5f with g). We wondered if Wnt3a treatment increased bone formation by blocking osteoclast activity, similar to the action of bisphosphonates. We used TRAP activity to identify osteoclasts in the injury site and found equivalent amounts of staining between the Wnt3a-treated and control sites (FIG. 5h, i). Thus, the enhanced bone regeneration elicited by Wnt3a liposomes did not appear to be attributable to a repression in osteoclast activity.

We gained more insights into the mechanism of Wnt3a action by examining the injury site using Xgal staining and in situ hybridization. We found that liposomal Wnt3a triggered a dramatic and sustained up-regulation in beta-galactosidase reporter activity in the bone marrow cavity at the injury site (compare control FIG. 5j with k). PCNA immunostaining was also increased (FIG. 5l, m), suggesting that liposomal Wnt3a stimulated the proliferation in a subset of bone marrow cells. Last, we noted strong, widespread expression of runx2, sox9, and osteocalcin in the injury site (FIGS. 5ns), which indicated more bone marrow cells had been recruited to an osteogenic fate as a result of Wnt3a exposure.

### Discussion

Damage to the skeleton triggers a reparative response in the body and our data indicate that one pathway involved in that reparative response is mediated by Wnts (FIG. 1 and Table 1). In response to skeletal damage, progenitor cells at the injury site begin to proliferate. Our data that at least one of the pathways involved in this injury-induced proliferation is mediated by Wnt signaling (FIG. 4). Once a sufficient population has been generated of skeletal progenitor cells begin to differentiate into osteoblasts. Although we do not have clues into how this transition between proliferation and differentiation occurs, our data show that Wnt signaling is a critical mediator of osteoblast differentiation in an injury site (FIGS. 3, 4). We further show that exogenous Wnt3a, delivered via a novel liposomal packaging method, leads to a dramatic enhancement in bone regeneration. This is achieved through at least two mechanisms. First, Wnt3a liposomes expedite the program of osteoblast differentiation at the wound site, as shown by the early induction of osteogenic genes and alkaline phosphatase activity (FIG. 5). Second, Wnt3a-treated injury sites had more PECAM positive endothelial cells (FIG. 5).

Our data show that a single injection of liposomal Wnt3a enhanced bone regeneration within a very short time frame. The rapidity of this response suggests that Wnt responsive cells were already present in the injury site at the time of drug delivery. We timed the delivery of Wnt3a liposomes to coincide with activation of the endogenous Wnt pathway following skeletal damage (FIG. 1), because we reasoned that the molecular machinery responsible for Wnt signaling would be fully functional by that time.

To our knowledge, this study represents the first in vivo use of purified Wnt proteins. Our initial attempts to deliver Wnt3a to the injury site were unsuccessful; neither purified protein by itself nor protein combined with a fibrin glue elicited any discernable effect in the wound environment. In sharp contrast, Wnt3a liposomes were highly efficacious at enhancing bone regeneration. A number of factors can contribute to the effectiveness of Wnt liposomes. Liposome packaging may stabilize and concentrate Wnt3a at the injury site. Wnt3a is modified by a palmitate residue, which is essential for its in vivo activity. The palmitate moiety itself has pronounced lipid affinity and during fabrication of the Wnt3a liposomes, it is probable that the palmitate associates with the liposome membrane. Consequently, Wnt3a may be effectively tethered to the liposome, which could prevent its clearance from the injury site.

Liposomes typically encapsulate molecules for in vivo delivery but our data show that the Wnt protein is not encapsulated. Rather, the protein is presented on the outer liposome membrane in its active conformation (FIG. 5a). In doing so, liposomes may imitate how many lipid modified proteins, including Wnts, are normally transported between cells. For example, lipid modified Wnts remain associated with a membrane even when they are being shuttled between cells, and there is evidence suggesting that Drosophila Wg is transported over many cell diameters in small vesicular structures. While the data supporting this vesicular transport mechanism still remain circumstantial, they do hint at an appealing opportunity: if the endogenous mechanism for transporting lipidated Wnts and other proteins to their target cells involves an association with lipid vesicle or raft, we may be able to commandeer this approach for the treatment of diseases or damage to the skeleton.

These data demonstrate that the Wnt pathway is a useful target for therapeutic interventions for the skeleton; and liposomes are an ideal delivery vehicle for lipid modified proteins, including the Wnts.

### Methods

Wnt reporter mice. BATgal and TOPgal transgenic mice were used as in vivo reporters of Wnt responsiveness. BATgal reporter mice carry a transgene which contains seven TCF binding motifs and the Xenopus siamois promoter. The TOPgal reporter cassette contains three copies of the TCF motif CCTTTGATC upstream of a minimal c-Fos promoter driving β-galactosidase gene expression. We used both strains to identify Wnt responding cells in the injury sites.

Generation of skeletal injuries. We have found that all of the steps comprising fracture healing are recapitulated by generating a simple trans-cortical defect, consisting of a 1.0 mm hole drilled through a single cortex of the tibia. The healing response in this defect is equivalent to a stabilized fracture and has a number of advantages over other models of closed and open fractures; first, morbidity to the animal is considerably reduced by limiting the scope of the skeletal injury. Second, the repair callus is much smaller and more spatially organized, which facilitates histomorphometric analyses. Third, trauma to surrounding tissues and infections are minimal in this model of skeletal tissue regeneration. All procedures were approved by the Stanford Committee on Animal Research.

Skeletally mature (10-12 weeks of age, male) mice were used for all studies. Following anesthesia and analgesia, the right leg was shaved and the skin cleansed. An incision was made over the proximal medial diaphysis and the anterior tibial muscle was divided until the medial surface of the tibia was exposed; the periosteum was preserved. Using a 1.0 mm drill bit, a hole was created that penetrated one cortex. The region was irrigated and the skin was closed using a non-absorbable suture.

During the recovery from anesthesia, all mice were kept under heating lamps to maintain constant body temperature. Their ability to reach food and water, their surgical site, activity, weight, appearance, and behavior were monitored. Mice were sacrificed at multiple time points that represented the inflammatory, hard callus, and remodeling phases of healing.

Molecular and cellular assays. Under RNAse-free conditions tibial callus tissues were harvested, the skin and outer layers of muscle were removed, and the tissues washed in lx PBS, 4° C. then fixed in 4% paraformaldehyde. Tissues were decalcified in 19% EDTA for 10-14 days and then prepared for paraffin embedding. Paraffin embedding followed our standard protocol 63 and sections were generated at an 8 μm thickness.

In situ hybridization: The relevant digoxigenin-labeled mRNA anti-sense probes were prepared from cDNA templates for wnt3a, dkk2, wnt2b, runx2, collagen type I, collagen type II, sox9, and osteocalcin. Sections were de-waxed, treated with proteinase K, and incubated in hybridization buffer containing the riboprobe. Probe was added at an approximate concentration of 0.25 μg/ml. Stringency washes of saline sodium citrate solution were done at 52° C., and further washed in maleic acid buffer with 1% Tween20. Slides were then treated with Anti-digoxigenin antibody (Roche). For color detection, slides were incubated in Nitro blue tetrazolium chloride and 5-bromo4-chloro-3-indolyl phosphate (Roche). After developing, the slides were cover-slipped with an aqueous mounting medium.

Immunohistochemistry: In general, tissue sections were de-waxed followed by immersion in H202/PBS, washed in PBS, incubated in Ficin (Zymed), treated with 0.1 M glycine, washed further, and then blocked in ovalbumin (Worthington) and 1% whole donkey IgG (Jackson Immunoresearch). Appropriate primary antibody was added and incubated overnight at 4° C., then washed in PBS. Samples were incubated with peroxidase-conjugated secondary antibody (Jackson Immunoresearch) for an hour and a DAB substrate kit (Vector Laboratories) was used to develop the color reaction. Some commonly used antibodies include proliferating cell nuclear antigen (PCNA) and platelet endothelial cell adhesion molecule 1 (PECAM-1). For Terminal transferase dUTP nick end labeling (TUNEL) staining of DNA strand breaks, sections were incubated in proteinase K buffer (20 μg/mL in 10 mM Tris pH 7.5) followed by TUNEL reaction mixture. (In situ Cell Death Detection Kit, Roche). Slides were viewed under a fluorescence microscope. For tartrate-resistant acid phosphatase (TRAP) staining, tissue sections were dewaxed and then treated with a TRAP staining kit (Sigma).

Quantification of cell proliferation: Quantification of PCNA positive cells was done using NIH Image J software. Four animals were used for each condition, Ad-Fc or Ad-Dkk1 treatment, and 10 random PCNA stained tissue sections were evaluated for each condition. Histology: Pentachrome and Aniline Blue staining were performed as described. Slides were mounted with Permount after dehydration with a series of ethanol and xylene.

β-galactosidase detection: Cells responsive to Wnt signaling express β-galactosidase, which can be detected by Xgal staining. For Xgal staining, tissues were fixed with 0.2% glutaraldehyde for 15 min and stained with Xgal overnight at 37° C. To detect the Xgal positive cells in the adenovirus treated samples, whole mount Xgal stained tissues were processed into paraffin blocks and sectioned. For liposome-treated samples, tissues were embedded in OCT followed by cryosectioning. Xgal staining on cryo-sections was performed in a similar fashion to whole mount.

Histomorphometric analyses. Tibiae were collected on either post-surgical d4 or d6 to analyze new bone and cartilage in the defect region. Tibiae were embedded in paraffin, sectioned longitudinally, and stained with Aniline Blue. In total, 4-7 animals were used for each condition (i.e., Ad-Fc, Ad-Dkk1, PBS liposome, or Wnt3a liposome; see text for details). The 1.0 mm circular mono-cortical defect was represented across approximately 40 tissue sections, each of which was 8 μm thick. Out of those 40 sections, 6-8 tissue sections were used for histomorphometric measurements. Each section was stained for mineralized tissue using Aniline Blue, and then photographed using a Leica digital imaging system (5× objective). The digital images were imported into Adobe Photoshop CS2. The region of interest encompassed 10₆ pixels and the number of Aniline blue stained pixels was determined using the magic wand tool (tolerance setting; 60, histogram pixel setting; cache level 1) by a single blinded investigator.

Adenovirus-mediated inhibition of Wnt signaling. All adenoviral constructs were generated previously 35. Adenoviruses expressing the soluble Wnt antagonist, Dkk1 and the murine IgG2a Fc fragment (Ad-Fc) were infected into 293 cells at MOI 1 in T175 tissue culture flasks (7.5×10₆ pfu). After 2 days, cells were collected, lysed, and precipitated by centrifugation. The supernatant was processed through freeze/thaw cycles and cell debris was removed by centrifugation. The supernatant was subjected to CsCl gradient centrifugation at 35,000 rpm for 20 h and the adenovirus band was harvested. Following sucrose dialysis the purified adenovirus was aliquoted and stored at −80° C. Wnt inhibition was achieved by either tail vein or local, sub-cutaneous injection of Ad-Dkk1 and the control Ad-Fc, and injuries were generated either immediately (in the case of tail vein injections) or after 48 h (in the case of local injections).

Liposomal packaging of Wnt3a protein. Wnt3a-liposomes were made using a 90:10:4 ratio of 1,2-dipalmitoyl-sn-Glycero-3-Phosphocholine (DPPC); 1-Myristoly-2-Palmitoyl-sn-Glycero-3-Phosphocholine (MPPC); 1,2-Distearoyl-sn-Glycero-3-Phosphoethanolamine-N-65Polyethylene Glycol)2000] (DSPE-PEG2000; Avanti Polar Lipids). One microgram of purified Wnt3a protein was packaged in the liposomes. Unilamellar vesicles were created by extruding the Wnt protein/liposome solution through a stack of two 100 nm polycarbonate membranes 40 times. After size extrusion, the Wnt3a liposomes were precipitated by ultracentrifugation at 28,000 rpm at 4° C. for 30 min. The Wnt3a liposome pellet was resuspended in 500 μl of DMEM supplemented with 10% fetal bovine serum (Hyclone). Control PBS liposomes were generated using identical conditions. A 30 μl aliquot of the Wnt3a liposomal preparation was delivered by injection to each site using a 29.5 gauge needle.

Western blot and RT-PCR. Calluses were collected from wild type (CD1) and BATgal mice 24 h post surgery, total RNA was extracted, and 1 μg of RNA was subjected to RT-PCR using a beta-galactosidase primer set. Western blot procedure was performed as previously described.

What is claimed is:

1. A method for the therapeutic delivery of a wnt polypeptide to an animal, the method comprising:
administering to said animal an effective dose of a wnt polypeptide comprising a lipid moiety, wherein the wnt protein is inserted in the non-aqueous phase of a lipid structure.

2. The method of claim 1, wherein the lipid structure is a liposome.

3. The method of claim 1, wherein the lipid structure is a micelle.

4. The method of claim 1, wherein the wnt polypeptide is a mammalian polypeptide.

5. The method of claim 1, wherein the wnt polypeptide is locally administered.

6. The method of claim 5, where the wnt polypeptide is administered by injection.

7. A pharmaceutical formulation comprising an effective dose of a wnt polypeptide comprising a lipid moiety, wherein the wnt protein is inserted in the non-aqueous phase of a lipid structure; and a pharmaceutically acceptable excipient.

8. The pharmaceutical formulation of claim 7, wherein the lipid structure is a liposome.

9. The pharmaceutical formulation of claim 7, wherein the lipid structure is a micelle.

10. The pharmaceutical formulation of claim 7, wherein the wnt polypeptide is a mammalian polypeptide.

11. A method of accelerating bone repair in a mammal, the method comprising:
administering to the mammal an effective dose of a wnt polypeptide comprising a lipid moiety, wherein the wnt protein is inserted in the non-aqueous phase of a lipid structure.

12. The method of claim 11, wherein the wnt polypeptide is injected at the site of a bone injury in the mammal.

13. The method of claim 12 where the wnt polypeptide is wnt 3A.

14. The method of claim 12 where the wnt polypeptide is administered within two days of the injury.

15. The method of claim 14 where the wnt polypeptide is administered for not more than two weeks.

16. The method of claim 11 where the mammal is provided with exogenous bone marrow cells.

17. The method of claim 16 where the bone marrow cells are treated ex vivo with an effective dose of wnt polypeptide.
